(19) United States
(12) Patent Application Publication
(10) Pub. No.: US 2021/0338862 A1
(43) Pub. Date: Nov. 4, 2021

(54) SELF-STERILIZING TOUCHPOINTS FOR LIGHT ELECTRIC VEHICLES

(71) Applicant: NEUTRON HOLDINGS, INC. DBA LIME, San Francisco, CA (US)

(72) Inventors: Peter PRIVITERA, San Francisco, CA (US); Joseph Daniel TAYLOR, San Francisco, CA (US)

(21) Appl. No.: 17/241,681

(22) Filed: Apr. 27, 2021

Related U.S. Application Data
(60) Provisional application No. 63/018,997, filed on May 1, 2020.

(51) Int. Cl. A61L 2/10; B62J 6/24; B62J 50/16; B62J 43/10; B62J 6/055

(52) U.S. Cl. CPC A61L 2/10; B62J 6/24; B62J 43/30; B62J 43/10; B62J 6/055; B62J 50/16

(57) ABSTRACT

This application describes a self-sterilizing handlebar and/or handlebar grip for a light electric vehicle. The handlebar described herein uses a light source, such as an ultra-violet light source, to transmit ultra-violet light within and/or through a handlebar and/or a handlebar grip. The ultra-violet light source transmits short-wavelength light to sterilize the handlebar grip.

### CROSS-REFERENCE TO RELATED APPLICATIONS

This application claims priority to and the benefit of U.S. Provisional Application No. 63/018,997 filed May 1, 2020, the disclosure of which is hereby incorporated by reference herein in its entirety.

### BACKGROUND

Providers of ridesharing platforms offer various transportation methods to enable individuals to reach a desired destination from their point of origin. In some cases, the ridesharing platform providers provide electric scooters and/or electric bikes that individuals can reserve and use for a period of time. However, due to the sheer number of electric bikes and electric scooters, it is time consuming and costly to clean the various electric vehicles.

### SUMMARY

The present application describes a handlebar and/or handlebar grip for a light electric vehicle. The handlebar and/or handlebar grip includes or are otherwise associated with components or systems that initiate a self-cleaning/sterilization process. In some examples, the self-cleaning/sterilization process (referred to herein as a “sterilization process”) uses a light source, such as an ultra-violet light source, to transmit ultra-violet light within and/or through a handlebar and/or a handlebar grip. Although a handlebar and a handlebar grip of the light electric vehicle are specifically mentioned, the systems, methods and examples described herein can be applied to various touchpoints or points of contact of a light electric vehicle including, but not limited to, a controller of a light electric vehicle, a seat of the light electric vehicle, a rechargeable battery of a light electric vehicle, a lock of the light electric vehicle and so on.

Accordingly, described herein is a method for initiating a sterilization process for a touchpoint of a light electric vehicle. This method includes determining when a reservation period for a light electric vehicle has ended. In response to determining the reservation period has ended, an internal component of the light electric vehicle will automatically initiate a sterilization process on a touchpoint of the light electric vehicle. The method also includes providing a notification that the sterilization process is complete.

The present application also describes a light electric vehicle that includes a handlebar defining an opening on an exterior surface. The light electric vehicle also includes an ultra-violet light source provided within an inner cavity of the handlebar and at least partially aligned with the slot. A handlebar grip is provided on the handlebar and the ultra-violet light source is adapted to initiate a sterilization process on the handlebar grip when the ultra-violet light source is illuminated.

This summary is provided to introduce a selection of concepts in a simplified form that are further described below in the Detailed Description. This summary is not intended to identify key features or essential features of the claimed subject matter, nor is it intended to be used to limit the scope of the claimed subject matter.

### BRIEF DESCRIPTION OF THE DRAWINGS

Non-limiting and non-exhaustive examples are described with reference to the following Figures.

FIG. 1 illustrates an example environment in which light electric vehicles are made available for reservation and use according to one or more examples.

FIG. 2A illustrates a handlebar of a light electric vehicle having a light pipe provided therein to perform a sterilization process according to one or more examples.

FIG. 2B illustrates a side view of the light pipe of FIG. 2A according to one or more examples.

FIG. 3A illustrates another example of a light source provided in handlebar of a light electric vehicle according to one or more examples.

FIG. 3B illustrates the handlebar of the light electric vehicle having one or more openings or slots to permit light to pass from the light source and into a handlebar grip of the light electric vehicle according to one or more examples.

FIGS. 4A-4B illustrate a system for initiating a sterilization process for a touchpoint of a light electric vehicle according to one or more examples.

FIG. 5 illustrates a method for initiating a sterilization process for a touchpoint of a light electric vehicle according to one or more examples.

FIG. 6 is a system diagram of a computing device that may be integrated or otherwise associated with a light electric vehicle according to one or more examples.

### DETAILED DESCRIPTION

In the following detailed description, references are made to the accompanying drawings that form a part hereof, and in which are shown by way of illustrations specific embodiments or examples. These aspects may be combined, other aspects may be utilized, and structural changes may be made without departing from the present disclosure. Examples may be practiced as methods, systems or devices. Accordingly, examples may take the form of a hardware implementation, an entirely software implementation, or an implementation combining software and hardware aspects. The following detailed description is therefore not to be taken in a limiting sense, and the scope of the present disclosure is defined by the appended claims and their equivalents.

Electric ridesharing vehicles, such as electric assist bicycles, electric scooters (also referred to herein as “light electric vehicles”), and mopeds, are widely used to transport individuals from various origins to various destinations. These light electric vehicles are typically provided by, or are otherwise associated with, a provider of a ridesharing platform. The providers of ridesharing platforms are also responsible for maintaining and cleaning the light electric vehicles. However, cleaning various touchpoints of the light electric vehicles, such as handlebars, handlebar grips, seats, locks, controllers etc., of the light electric vehicle can be a time consuming process. Additionally, since a light electric vehicle can essentially be picked up, ridden and dropped off at any number of different locations and at different times during the day, ensuring these touchpoints are regularly cleaned is a daunting task.

Accordingly, the present application describes various systems, components, and methods that may be used by or associated with a light electric vehicle to clean various touchpoints of the light electric vehicle. Although the examples described herein are specifically directed to a handlebar and/or handlebar grip of the light electric vehicle, similar systems, components, and methods may be applied to various other touchpoints or points of contact of a light electric vehicle including, but not limited to, a controller of a light electric vehicle, a seat of the light electric vehicle, a rechargeable battery of a light electric vehicle, a lock of the light electric vehicle and so on.

In some examples, a handlebar of the light electric vehicle may include an internal component having various different light sources. In the examples described below, the internal component is described as a light pipe. The light pipe may include a single type of light source or many different types of light sources. For example, the light pipe may include light sources that emit ultra-violet light and light sources that emit visible light.

In the examples described herein, the light pipe is disposed within a cavity or chamber of the handlebar. For example, the interior portion of the handlebar may be hollow which enables the light pipe to be placed therein. As indicated above, the light pipe may include one light source or many light sources. The light sources may include ultra-violet light sources that emit UV-C light or other short-wavelength light. When the ultra-violet light source is illuminated, it emits UV-C light through one or more slots or channels defined by the handlebar. The emitted light may then be transmitted into the handlebar grips of the light electric vehicle to initiate a sterilization process.

In some example, the handlebar grips may be made of a transmissive material or color and/or have an opacity that allows or otherwise enables the transmission of light through the handlebar grip. The handlebar grip may also include or otherwise define one or more slots or channels that enables the transmitted light to pass into and/or through the handlebar grip.

In some examples, the light pipe may also include other light sources such as RGB LEDs or other such light sources that produce visible light. The visible light may be used to indicate whether a sterilization process is in progress or is complete. For example, the RGB LEDs may emit a red light while a sterilization process is ongoing and may emit a green light when the sterilization process is complete.

The RGB LEDs may also be used to provide other operating conditions of the light electric vehicle. For example, these light sources may indicate that the light electric vehicle is turning, is available for reservation or use, is unavailable for reservation or use and so on.

In another example, an ultra-violet light source and/or other light sources (e.g., RGB LEDs) may be positioned on an edge portion of a handlebar or handlebar grip. When these light sources are illuminated, they provide the same or similar benefits as described above.

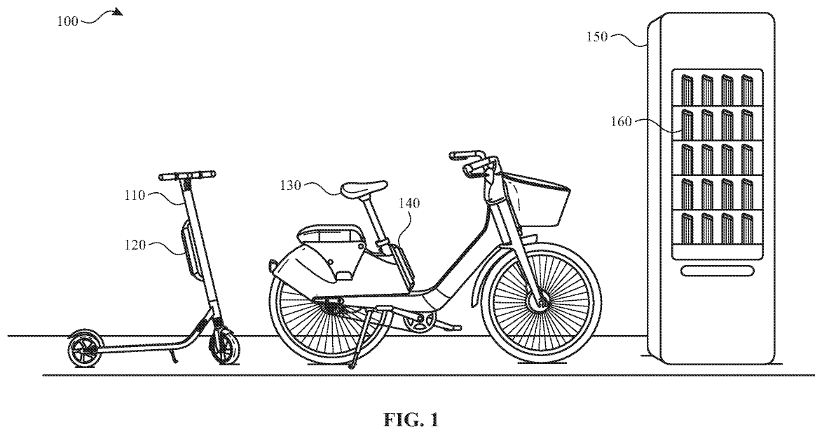

These and other examples will be shown and described with reference to FIGS. 1-6 below.

FIG. 1 illustrates an example environment 100 in which aspects of the present disclosure may be practiced. As illustrated, environment 100 includes an electric scooter(s) 110, an electric bicycle(s) 130, and a rechargeable battery kiosk(s) 150. It will be appreciated that the electric scooter 110 and the electric bicycle 130 are provided as example light electric vehicles and that, in other examples, aspects described herein apply to other types of light electric vehicles.

As described herein, the environment 100 includes a network service that receives information from the electric scooter 110 and/or the electric bicycle 130 (also referred to herein as light electric vehicles) over a network communication channel (e.g., one or more networks, the Internet, etc.). The information enables an individual, using a client application executing on a computing device, to locate, request, and/or reserve (e.g., rent or borrow for a duration of time) one or more light electric vehicles.

In some examples, the network service includes one or more computing systems or servers that are remote from the computing device of the individual and the light electric vehicles. The one or more computing systems includes an application programming interface (API) that enables the one or more computing systems to receive information from, send information to, and otherwise interact with the computing device, the light electric vehicles 110, 130 and/or the rechargeable battery kiosk(s) 150.

For example, the client application executing on the computing device of the individual receives, from the network service over the network communication channel, information about a location of one or more of the light electric vehicles. The location of each of the light electric vehicles can then be provided on a user interface of the client application.

In one example, the user interface of the client application includes a map that displays a determined location of the individual and/or a determined location of the light electric vehicles. In some examples, the determined location of the individual and/or the determined location of the light electric vehicles is based, at least in part, on Global Positioning System (GPS) data (or other location information) received by the network service over the network communication channel.

The user interface of the client application displays the location information of the individual and the light electric vehicles as different icons (or other such visual representations). Once the location information is displayed, the individual may select an icon representing a type of light electric vehicle (e.g., an icon for an electric scooter 110 or an icon for an electric bicycle 130). The user interface of the client application then generates or determines a route (e.g., provides directions) from the individual's current location to the selected light electric vehicle. Selection of one of the icons may also enable the individual to reserve (e.g., place a hold on) the light electric vehicle (to ensure that the light electric vehicle will be at the determined location when the individual arrives), rent the light electric vehicle and/or borrow the light electric vehicle for a period of time.

Each light electric vehicle and/or the network service also includes a location tracking system that tracks, receives and/or determines a location of each light electric vehicle as they are used. In some examples, the location tracking system tracks the location information of the light electric vehicle in real-time or substantially real-time. In other examples, the location tracking system determines the location information of the light electric vehicle at periodic intervals (e.g., every minute, every five minutes, every ten minutes, etc.). In yet other examples, the location tracking system may track the location of the light electric vehicle in real-time or substantially real-time when the light electric vehicle is reserved, rented or otherwise used by an individual and may track location information at periodic intervals when the light electric vehicle has been reserved or is otherwise not in use.

The one or more computing systems of the network service also include one or more databases that store information about each of the light electric vehicles and/or the rechargeable battery kiosk(s) 150. For example, the one or more databases may store location information for each light electric vehicle and/or the rechargeable battery kiosk(s) 150, rechargeable battery status information for rechargeable batteries used by each light electric vehicle and/or in the rechargeable battery kiosk(s) 150, rechargeable battery kiosk information (e.g., the number of rechargeable batteries housed by the rechargeable battery kiosk 150), and/or light electric vehicle status information (e.g., how many times the light electric vehicle has been reserved, whether the light electric vehicle is damaged, whether the light electric vehicle should be serviced, etc.).

The one or more databases may also store information about the individual. This information may include a profile of the individual (e.g., username, contact information, etc.) security credentials of the individual (e.g., a password), historical usage data, payment information and the like. The one or more databases may also store information about the light electric vehicle including one or more results of a brake system inspection process.

The one or more computing systems of the network service may also include a matching system. The matching system receives, manages or otherwise handles various requests from the individual. The requests may include light electric vehicle rental requests and light electric vehicle reservation requests. For example, when a light electric vehicle use request is received from the client application executing on the individual's computing device, the matching system may communicate with the location tracking system and determine which light electric vehicle should be matched with or otherwise assigned to the requesting individual.

The one or more computing systems of the network service may also include a payment system that processes payment information of the individual and/or distributes received incentives to the individual. For example, when an individual rents and uses a light electric vehicle, the individual may be charged for the usage based on a duration of use and/or a travel distance. Once the individual has finished using the light electric vehicle (e.g., by arriving at her intended destination, a check-in point, a battery kiosk 150, etc.), the payment system may automatically process the payment information of the individual Likewise, if the individual is offered an incentive (e.g., for parking the light electric vehicle in a certain location), the payment system may apply or otherwise provide the incentive to the individual.

As discussed above, the environment 100 includes one or more light electric vehicles including, but not limited to, an electric scooter 110 and an electric bicycle 130. In examples, the electric scooter 110 includes vehicle components (e.g., wheels, axles, baseboard, handlebar, braking mechanisms, etc.), one or more electric motors, control systems, sensors, speakers, and/or lights, which may be powered by a rechargeable battery. The rechargeable battery may be secured to the electric scooter 110 by a battery holster 120.

Likewise, and in some examples, the electric bicycle 130 includes vehicle components (e.g., wheels, axles, chains, gear ratios, bicycle seat, handlebar, bicycle frame, braking mechanisms, etc.), one or more electric motors, control systems, sensors, speakers, and/or lights, which may also be powered by a rechargeable battery. The rechargeable battery may be secured to the electric bicycle 130 by a battery holster 140.

The control system of the electric scooter 110 and/or the electric bicycle 130 may include a controller or control mechanism such as described herein. As such, the control system may manage the power output to the one or motors, provides a visual indication as to a charge level of the rechargeable battery in the battery holster 120, and/or communicates directly (e.g., via Wi-Fi, Bluetooth, etc.) or indirectly (e.g., via one or more remote computing devices, one or more networks, the Internet, etc.) with the computing device of the individual and/or with the network service.

Example communications include, but are not limited to, initiating locking or unlocking of the electric scooter 110 or the electric bicycle 130 (e.g., initiating or ending a travel session), initiating a battery swap to exchange a rechargeable battery in the battery holster 120 or the battery holster 140 with one in a rechargeable battery kiosk 150, determining a location and/or status information of the electric scooter 110 or the electric bicycle 130, and determining a location of a rechargeable battery and/or a rechargeable battery kiosk 150. Lights, speakers, and/or other output devices of the electric scooter 110 or the electric bicycle 130 may be used to provide an indication as to the location of the electric scooter 110 or the electric bicycle 130 or as an anti-theft mechanism, among other examples.

As shown in FIG. 1, each light electric vehicle includes a battery holster. For example, the battery holster 140 is affixed to the seat tube of the electric bicycle 130, while the battery holster 120 is illustrated as being affixed to the handlebar column of the electric scooter 110. It will be appreciated that the locations of the battery holsters 120 and 140 are provided as examples, and that a battery holster may be positioned in a variety of alternate locations in other examples. For example, the battery holster 140 may be affixed to the handlebar column or the cross bar of the electric bicycle 130. As another example, the battery holster 120 may be affixed to the deck or located near the rear of the electric scooter 110.

The battery holsters 120 and 140 are each operable to receive a rechargeable battery. For example, an individual may operate a light electric vehicle for a period of time and then determine that the rechargeable battery in use by the light electric vehicle needs to be recharged. In some instances, the light electric vehicle, or the rechargeable battery itself, may communicate current battery charge information for the rechargeable battery to the computing device of the individual. In another example, the rechargeable battery and/or battery holster 120 and 140 may include a visual indicator to display the charge level of the rechargeable battery. In yet another example, a control mechanism coupled to the light electric vehicle may include one or more indicators that reflect the charge level of the rechargeable battery. As an addition or an alternative, the light electrical vehicle, the control mechanism and/or the rechargeable battery itself, may communicate current battery charge information for the rechargeable battery to the network service, which can provide battery information to the computing device of the individual. When this occurs, the individual may be directed to a rechargeable battery kiosk 150. For example, the network service can transmit data, over one or more networks, to the computing device and/or the control mechanism to cause the computing device and/or control mechanism to display information about a particular rechargeable battery kiosk 150 that individual could travel to in order to initiate a rechargeable battery exchange.

When the individual arrives at the rechargeable battery kiosk 150, the individual may exchange the light electric vehicle's current battery with another rechargeable battery housed by the rechargeable battery kiosk 150, thereby enabling the light electric vehicle to continue or resume operation. In some instances, the individual can use the client application executing on the computing device of the individual to locate and/or select a rechargeable battery kiosk 150, receive directions to the rechargeable battery kiosk 150, and initiate a rechargeable battery exchange with the rechargeable battery kiosk 150 when the individual arrives at its location. In another example, a selectable element (e.g., a button) on the control mechanism may enable the individual to initiate a rechargeable battery exchange.

According to examples, when the rechargeable battery exchange is initiated, the control system of the light electric vehicle may enable the rechargeable battery 160 to be removed from a battery holster, such as battery holster 120 or 140. The rechargeable battery 160 may then be exchanged for a different rechargeable battery 160 housed by the rechargeable battery kiosk 150. The rechargeable battery 160 may subsequently be inserted into the battery holster of the light electric vehicle.

The rechargeable battery kiosk 150 stores and charges a set of rechargeable batteries 160. Each rechargeable battery 160 in the set can be used by both the electric scooter 110 and the electric bicycle 130. In some examples, multiple rechargeable battery kiosks 150 are located within a city, county, or other geographic region. For example, one rechargeable battery kiosk may be located in or otherwise associated with a first geographic area within a geographic region and another rechargeable battery kiosk may be located in or otherwise associated with a second geographic area within the geographic region.

Thus, when an individual is traveling through the geographic region on a light electric vehicle and wants or needs to exchange the light electric vehicle's current rechargeable battery for one that has more charge, the individual may be directed (e.g., via the client application executing on the individual's computing device) to the rechargeable battery kiosk 150 associated with the geographic region. When the individual arrives at the rechargeable battery kiosk 150, the individual can exchange their current rechargeable battery for one that is fully charged or substantially fully charged. This enables the individual to travel using a light electric vehicle across distances that may otherwise not be possible using the power provided by one charge of a rechargeable battery.

In some examples, the rechargeable battery kiosk 150 comprises a control system that communicates directly or indirectly with a computing device of the individual and/or the control mechanism of the light electric vehicle when performing the rechargeable battery exchange such as described above. In some examples, the control system communicates with a remote computing device(s), e.g., that implements the network service, using a connection to one or more networks, such as a Wi-Fi network and/or a cellular network. The rechargeable battery kiosk 150 may receive and/or report rechargeable battery status information to a remote computing device(s). The battery status information can include, but is not limited to, battery charge levels, battery health, an amount of rechargeable batteries currently available at the rechargeable battery kiosk, and/or usage demand statistics.

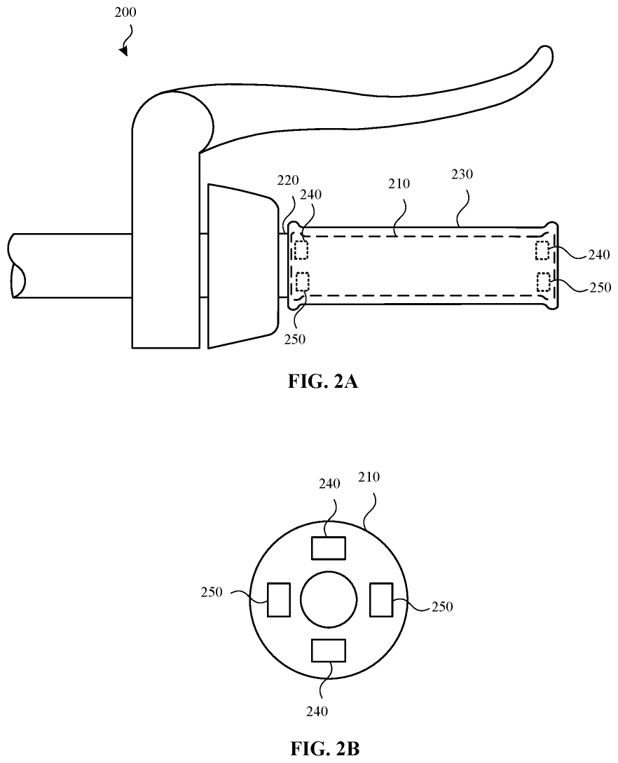

FIG. 2A illustrates a handlebar 200 of a light electric vehicle having a light pipe 210 provided therein to perform a sterilization process according to one or more examples. The handlebar 200 may be used with a light electric vehicle such as an electric scooter 110 (FIG. 1) and an electric bicycle 130 (FIG. 1).

The handlebar 200 may include a handlebar portion 220 over which a handlebar grip 230 may be placed. The handlebar grip 230 may be made of silicon, rubber, or other material that enables light to be transmitted or passed therethrough. The handlebar grip 230 may also have a particular color and/or opacity which enables or otherwise permits light to be diffused through the handlebar grip 230.

In some examples, the handlebar grip 230 may include textures. In other examples, the handlebar grip may be smooth. In yet other examples, the handlebar grip 230 may include one or more slots or channels that assist in the transmission of light (both visible light and short-wavelength light) through the handlebar grip 230. The handlebar grip 230 may be removable from the handlebar portion 220 of the handlebar 200. Thus, when the handlebar grip 230 becomes damaged or worn, it can be replaced with a new handlebar grip 230.

The light pipe 210 may include a first light source 240 and a second light source 250. As shown in FIG. 2A, the first light source 240 and the second light source 250 may be positioned at proximal and distal ends of the light pipe 210. Although the first light source 240 and the second light source 250 are positioned at proximal and distal ends of the light pipe 210, each of these light sources may be positioned at different locations. In other examples, the first light source 240 may be positioned at a proximal end of the light pipe 210 and the second light source 250 may be positioned at a distal end of the light pipe 210 or vice versa.

In some examples, the first light source 240 is an ultra-violet light source that transmits UV-C light or other short-wavelength light that may be used to clean and/or sterilize the handlebar grip 230. The second light source 250 may be a light source that emits visible light such as, for example RGB LEDs or other such light sources. The second light source 250 may be used to provide a status notification and/or an operating condition notification of the light electric vehicle to individuals.

For example, when the first light source 240 is emitting UV-C light to sterilize the handlebar grip 230, the second light source 250 may emit colored visible light (e.g., red light) to indicate a sterilization process is ongoing and/or indicate the light electric vehicle is currently unavailable for reservation and/or use. Once the sterilization process is complete, the first light source 240 may stop emitting UV-C light and the second light source 250 may emit a different colored visible light (e.g., a green light) to indicate that one or more touchpoints of the light electric vehicle has undergone a sterilization process and/or the light electric vehicle can be reserved and used.

The second light source may also emit light based on detected or determined environmental conditions and/or based on detected or determined operating conditions. For example, if one or more sensors on the light electric vehicle or a light electric vehicle management system associated with the light electric vehicle determines the light electric vehicle is being used at night, the second light source may be illuminated to help make the light electric vehicle more visible to others on the road. Additionally, if one or more sensors on the light electric vehicle or a light electric vehicle management system associated with the light electric vehicle determines that the light electric vehicle is turning, the second light source may be illuminated to indicate a turn. In other examples, a switch or lever on the handlebar 200 may be actuated to illuminate the second light source 250.

FIG. 2B illustrates a side view of the light pipe 210 of FIG. 2A according to one or more examples. As shown in FIG. 2B, the light pipe 210 may have a circular configuration. In some examples, the circular configuration enables the light pipe 210 to be inserted into an inner cavity of the handlebar portion 220. Once inserted, the first light source 240 and the second light source 250 may be arranged such that light is emitted into a proximal end and/or a distal end of a handlebar grip 230 once the handlebar grip 230 has been coupled to the handlebar portion 220. For example, the first light source 240 and/or the second light source 250 may extend from a proximal end and/or a distal end of the handlebar portion 220 such that each of the light sources can emit light in and/or through the handlebar grip 230.

In another example, the light pipe 210 may be placed over an outer surface of the handlebar portion 220. For example, the light pipe 210 may define a hole in the center or otherwise have a “sleeve” configuration that enables the light pipe 210 to be placed over the outer surface of the handlebar portion 220. Once the light pipe 210 has been placed over the outer surface of the handlebar portion 220, the handlebar grip 230 may be placed over the light pipe 210. In some examples, the light pipe 210, the handlebar portion 220 and/or the handlebar grip 230 may include a locking device or mechanism that secures the light pipe 210 to the handlebar portion 220 and/or the handlebar grip 230 to prevent or inhibit movement of the light pipe 210 and/or the handlebar grip 230.

Although FIG. 2B shows the first light source 240 and the second light source 250 being arranged in a particular configuration and with a particular quantity, the light pipe 210 may include any number of light sources arranged in various configurations.

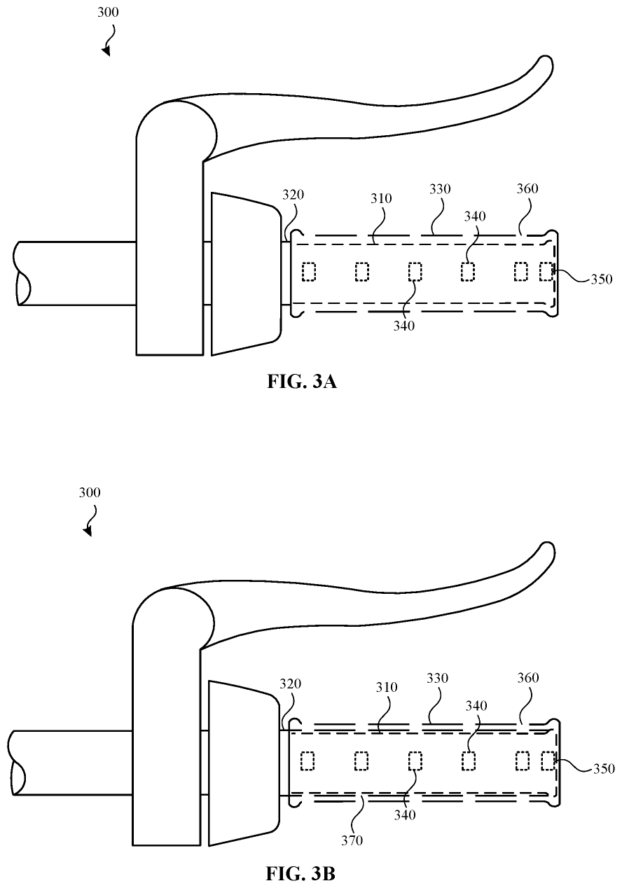

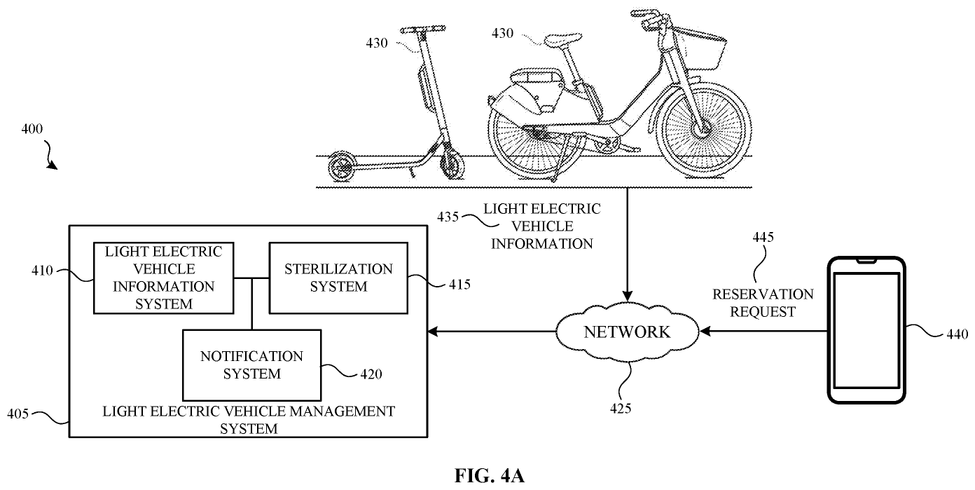

FIG. 3A illustrates another example of a light pipe 310 provided in handlebar 300 of a light electric vehicle according to one or more examples. In the example shown in FIG. 3A, the light pipe 310 may be provided or is otherwise positioned within a cavity of a handlebar portion 320 of the handlebar 300. In another example, the light pipe 310 may be provided on an outer surface of the handlebar portion 320.

The light pipe 310 may include a number of first light sources 340 positioned along an axis (or other position) of the light pipe 310. In the example shown in FIG. 3A, the light pipe 310 includes five first light sources 340. Although five first light sources 340 are shown, the light pipe 310 may have any number of first light sources 340. Like the example previously described, each of the first light sources 340 may be ultra-violet light sources that emit UV-C light or other such short-wavelength light in order to sterilize the handlebar grip 330.

The light pipe 310 may also include one or more second light sources 350. The one or more second light sources 350 may be positioned on a distal end of the light pipe 310. In another example, the one or more second light sources 350 may be positioned at different locations along an axis (or other position) of the light pipe 310. Each of the first light sources 340 and the one or more second light sources 350 function in a similar manner as the first light source 240 and the second light source 250 described previously with respect to FIG. 2A and FIG. 2B.

However, in order to help ensure that light from the first light sources 340 and/or the one or more second light sources are transmitted in and/or through the handlebar grip 330, the handlebar grip 330 may include or otherwise define one or more openings, channels, grooves etc. (referred to as surface features 360). These surface features 360 may enable light to be transmitted and/or diffused entirely though (or substantially entirely through) the handlebar grip 330.

Although specific surface features 360 are mentioned, other surface features may be used. Example surface features include textures, depressions, cavities, holes, protrusions, channels, etc. In some examples, these surface features 360 may be positioned at certain locations/positions on the handlebar grip 330. In other examples, the surface features 360 may be provided on all or substantially all of the handlebar grip 330. In some examples, the surface features 360 of the handlebar grip 330 may be positioned such that they are aligned with one or more of the first light sources 340 and/or the one or more second light sources 350.

FIG. 3B illustrates the handlebar 300 of the light electric vehicle having one or more openings or slots 370 to permit light to pass from the first light sources 340 and/or the one or more second light sources 350 into a handlebar grip 330 of the light electric vehicle. The one or more slots 370 may be defined by the handlebar portion 320 and may extend from an outer surface of the handlebar portion 320 to an inner surface of the handlebar portion 320 such that light can pass through the handlebar portion 320.

In some examples, the light pipe 310 may be positioned within the handlebar portion 320 such that the various light sources may be aligned or substantially aligned with the slots 370 of the handlebar portion 320. Additionally, the surface features 360 of the handlebar grip 330 may be aligned or substantially aligned with the slots 370 of the handlebar portion 320.

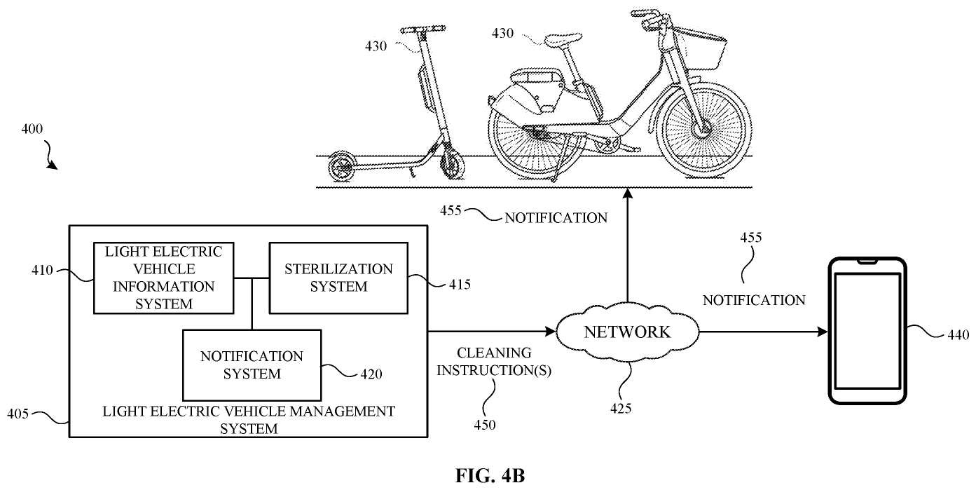

FIGS. 4A-4B illustrate a system 400 for initiating a sterilization process for a touchpoint of a light electric vehicle according to one or more examples. As shown in FIG. 4A, the system 400 includes a light electric vehicle management system 405. The light electric vehicle management system 405 may be used to determine when a touchpoint sterilization process should occur for a light electric vehicle 430 and/or may send instructions to a light electric vehicle 430 to initiate a touchpoint sterilization process. The touchpoint sterilization process may involve causing an internal component of the light electric vehicle, such as for example, a light pipe, having various light sources to initiate a sterilization process on a handlebar grip or other touchpoint of a light electric vehicle such as previously described with respect to FIGS. 2A-3B.

In some examples, and in an effort to effectively clean or otherwise initiate a sterilization process, the light electric vehicle management system 405 and/or a light electric vehicle 430 may determine when a reservation period for the light electric vehicle 430 has ended. In another example, the light electric vehicle management system 405 and/or the light electric vehicle 430 may determine to initiate a sterilization process when a reservation request for a light electric vehicle is received.

For example, a computing device 440 associated with an individual that is seeking to use or reserve a particular light electric vehicle 430 may provide, over a network 425, a reservation request 445 to the light electric vehicle management system 405 to reserve and use the particular light electric vehicle 430. In response to receiving the reservation request 445, the light electric vehicle management system 405 may determine, along with other systems associated with the light electric vehicle management system 405, that a sterilization process should be initiated.

In some examples, a duration of the sterilization process may be based, at least in part, on a location of the light electric vehicle 430, environmental conditions of an area in which the light electric vehicle 430 is located, the type of materials used on the light electric vehicle 430 (e.g., the materials used for the handlebar grips), and so on.

Accordingly, the light electric vehicle management system 405 may receive, over the network 425, light electric vehicle information 435 from one or more light electric vehicles 430. The light electric vehicle information 435 may include location information of the light electric vehicle 430, make and model information of the light electric vehicle 430, information about the materials of one or more components of the light electric vehicle 430, reservation information associated with the light electric vehicle 430 (e.g., whether the light electric vehicle 430 is currently being used, whether a reservation period for the light electric vehicle 430 has ended, whether the light electric vehicle 430 has been reserved but a use period has not started, etc.), information regarding the occurrence or frequency of a sterilization process for the light electric vehicle 430 and so on.

As the light electric vehicle management system 405 receives the light electric vehicle information 435, the light electric vehicle information 435 may be stored in or otherwise provided to a light electric vehicle information system 410. The light electric vehicle information system 410 may then determine, based on the light electric vehicle information 435, whether to initiate a sterilization process.

For example, if the light electric vehicle information 435 indicates that a reservation period of the light electric vehicle has ended, the light electric vehicle management system 405 (or the light electric vehicle information system 410) may provide this information to the sterilization system 415. The sterilization system 415 may then determine whether to initiate a sterilization process. As indicated above, the duration of the sterilization process may be based on a number of different factors including current environmental conditions in a location in which the light electric vehicle 430 is located, the type of handlebar grips provided on the light electric vehicle 430 and so on.

For example, if the light electric vehicle 430 is located in an area that is sunny or has a hotter climate, a sterilization process may be shorter when compared to a sterilization process for a light electric vehicle 430 that is located in colder climates Likewise, if the handlebar grips of the light electric vehicle are opaque, the sterilization process may be longer when compared to handlebar grips that are comparatively less opaque.

The light electric vehicle management system 405 may also include a notification system 420. The notification system 420 may be used to provide a notification to an internal component (e.g., light pipe 210 (FIG. 2A)) to illuminate various light sources to provide a notification that a sterilization process is underway or has been completed.

For example and turning to FIG. 4B, once the light electric vehicle information 435 has been received by the light electric vehicle management system 405, and the sterilization system 415 has determined the parameters of the sterilization process (e.g., whether to initiate a sterilization process, the duration of the sterilization process, etc.), the light electric vehicle management system 405 may transmit or otherwise provide cleaning instructions 450 to the light electric vehicle 430. Upon receipt of the cleaning instructions 450, an internal component, such as a light pipe of the light electric vehicle may illuminate one or more light sources (e.g., first light source 240 (FIG. 2A)) provided on the light electric vehicle 430 such as previously described.

The notification system 420 of the light electric vehicle management system 405 may also transmit or otherwise provide notification instructions 455 to the light electric vehicle 430 and/or a computing device 440 associated with an individual that a sterilization process is occurring or has been completed. For example, when the notification 455 is provided to the light electric vehicle 430, the internal component may illuminate a second light source (e.g., second light source 250 (FIG. 2A)) in a first color (e.g., red) to indicate that a sterilization process has been initiated. As such, the notification indicates that the light electric vehicle 430 is not available for reservation or use. However, once the sterilization process is complete, the internal component may illuminate the second light source in a second color (e.g., green or white) indicating that a sterilization process is complete and/or that the light electric vehicle 430 is available for reservation and use.

Likewise, the notification 455 provided to the computing device 440 may indicate whether a selected light electric vehicle 430 has recently undergone a sterilization process or is in the process of undergoing a sterilization process. In some examples, input received on the computing device 440 and/or on a controller of the light electric vehicle 430 may also cause a sterilization process to commence.

Although FIGS. 4A-4B illustrate the light electric vehicle management system 405 being remote from the light electric vehicles 430, the light electric vehicle management system 405, or components and systems of the light electric vehicle management system 405, may be integrated with the light electric vehicle 430. Additionally, the various handlebar and light pipe configurations described with respect to FIGS. 2A-3B may be integrated with the light electric vehicles 430.

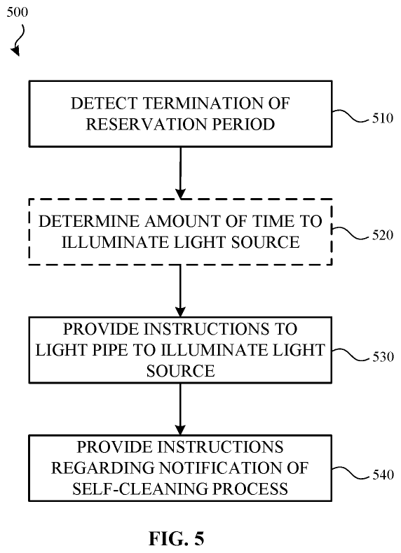

FIG. 5 illustrates a method 500 for initiating a sterilization process for a touchpoint of a light electric vehicle according to one or more examples. The method 500 may be used by a light electric vehicle management system, such as light electric vehicle management system 405 described with respect to FIG. 4A-FIG. 4B.

Method 500 begins when a light electric vehicle management system detects (510) that a reservation period for a light electric vehicle has ended. Once the reservation period has ended, the light electric vehicle management system may determine (520) an amount of time to illuminate a light source of a light pipe provided on the light electric vehicle. In some examples, the light pipe may include a number of different light sources including ultra-violet lights that emit UV-C light and RGB LEDs that emit visible light. The amount of time may be based on a number of factors including a material of one or more components of the light electric vehicle that is being sterilized, environmental conditions in the area in which the light electric vehicle is located and so on.

When the amount of time is determined, lighting instructions are provided (530) to the light pipe of the light electric vehicle. The lighting instructions cause the light pipe to illuminate one or more ultra-violet light sources for the determined amount of time. The light electric vehicle management system may also provide (540) notification instructions to the light pipe of light electric vehicle. The notification instructions may include instructions that one or more visible lights be illuminated to indicate either that a sterilization process is ongoing (and therefore the light electric vehicle is not available for reservation or use) or is complete (and therefore the light electric vehicle is available for reservation and use).

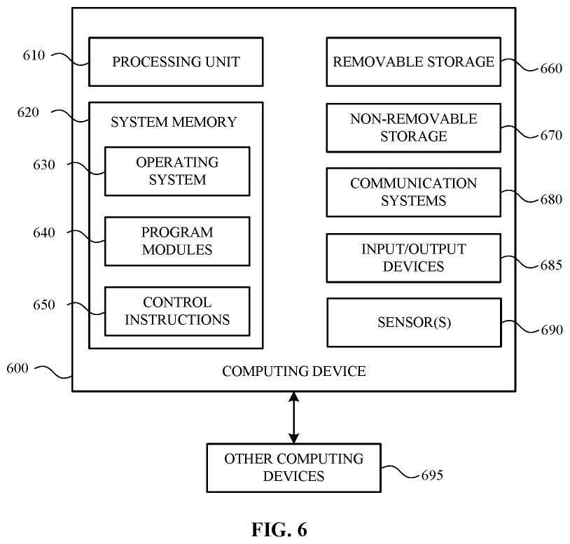

FIG. 6 is a system diagram of a computing device 600 according to an example. The computing device 600 may be integrated with or associated with a light electric vehicle and/or the light electric vehicle brake inspection device described herein. As shown in FIG. 6, the physical components (e.g., hardware) of the computing are illustrated and these physical components may be used to practice the various aspects of the present disclosure.

The computing device 600 may include at least one processing unit 610 and a system memory 620. The system memory 620 may include, but is not limited to, volatile storage (e.g., random access memory), non-volatile storage (e.g., read-only memory), flash memory, or any combination of such memories. The system memory 620 may also include an operating system 630 that controls the operation of the computing device 600 and one or more program modules 640. The program modules 640 may be responsible for gathering or determining light electric vehicle information. The memory 620 may also store and/or provide system control instructions 650 that causes the various light sources of a light pipe to be illuminated such as described. A number of different program modules and data files may be stored in the system memory 620. While executing on the processing unit 610, the program modules 640 may perform the various processes described above.

The computing device 600 may also have additional features or functionality. For example, the computing device 600 may include additional data storage devices (e.g., removable and/or non-removable storage devices) such as, for example, magnetic disks, optical disks, or tape. These additional storage devices are labeled as a removable storage 660 and a non-removable storage 670.

Furthermore, examples of the disclosure may be practiced in an electrical circuit comprising discrete electronic elements, packaged or integrated electronic chips containing logic gates, a circuit utilizing a microprocessor, or on a single chip containing electronic elements or microprocessors. For example, examples of the disclosure may be practiced via a system-on-a-chip (SOC) where each or many of the components illustrated in FIG. 6 may be integrated onto a single integrated circuit. Such a SOC device may include one or more processing units, graphics units, communications units, system virtualization units and various application functionality all of which are integrated (or “burned”) onto the chip substrate as a single integrated circuit.

When operating via a SOC, the functionality, described herein, may be operated via application-specific logic integrated with other components of the computing device 600 on the single integrated circuit (chip). The disclosure may also be practiced using other technologies capable of performing logical operations such as, for example, AND, OR, and NOT, including but not limited to mechanical, optical, fluidic, and quantum technologies. In addition, examples of the disclosure may be practiced using a computing device associated with or integrated with the electric vehicle and/or in any other circuits or systems.

The computing device 600 may include one or more communication systems 680 that enable the electric vehicle to communicate with rechargeable batteries, other computing devices 695, a network service and the like. Examples of communication systems 680 include, but are not limited to, wireless communications, wired communications, cellular communications, radio frequency (RF) transmitter, receiver, and/or transceiver circuitry, a Controller Area Network (CAN) bus, a universal serial bus (USB), parallel, serial ports, etc.

The computing device 600 may also have one or more input devices and/or one or more output devices shown as input/output devices 685. These input/output devices 685 may include a keyboard, buttons, switches, a sound or voice input device, haptic devices, a touch, force and/or swipe input device, a display, speakers, etc. The aforementioned devices are examples and others may be used.

The computing device 600 may also include one or more sensors 690. The sensors may be used to detect or otherwise provide information about the operating condition of the computing device 600. In other examples, the sensors 690 may provide information about a light electric vehicle, environmental conditions in an area in which the light electric vehicle is located, etc.

The term computer-readable media as used herein may include computer storage media. Computer storage media may include volatile and nonvolatile, removable and non-removable media implemented in any method or technology for storage of information, such as computer readable instructions, data structures, or program modules.

The system memory 620, the removable storage 660, and the non-removable storage 670 are all computer storage media examples (e.g., memory storage). Computer storage media may include RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other article of manufacture which can be used to store information and which can be accessed by the computing device 600. Any such computer storage media may be part of the computing device 600. Computer storage media does not include a carrier wave or other propagated or modulated data signal.

Communication media may be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media.

The description and illustration of one or more aspects provided in this application are not intended to limit or restrict the scope of the disclosure as claimed in any way. The aspects, examples, and details provided in this application are considered sufficient to convey possession and enable others to make and use the best mode of claimed disclosure. The claimed disclosure should not be construed as being limited to any aspect, example, or detail provided in this application. Regardless of whether shown and described in combination or separately, the various features (both structural and methodological) are intended to be selectively rearranged, included or omitted to produce an embodiment with a particular set of features. Having been provided with the description and illustration of the present application, one skilled in the art may envision variations, modifications, and alternate aspects falling within the spirit of the broader aspects of the general inventive concept embodied in this application that do not depart from the broader scope of the claimed disclosure.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof means any connection or coupling, either direct or indirect, between two or more elements; the coupling or connection between the elements can be physical, logical, or a combination thereof. Additionally, the words “herein,” “above,” “below,” and words of similar import, when used in this application, refer to this application as a whole and not to any particular portions of this application. Where the context permits, words in the above Detailed Description using the singular or plural number may also include the plural or singular number respectively. The word “or,” in reference to a list of two or more items, covers all of the following interpretations of the word: any of the items in the list, all of the items in the list, and any combination of the items in the list.

Several implementations of the disclosed technology are described above in reference to the figures. The computing devices on which the described technology may be implemented can include one or more central processing units, memory, input devices (e.g., keyboards and pointing devices), output devices (e.g., display devices), storage devices (e.g., disk drives), and network devices (e.g., network interfaces). The memory and storage devices are computer-readable storage media that can store instructions that implement at least portions of the described technology. In addition, the data structures and message structures can be stored or transmitted via a data transmission medium, such as a signal on a communications link. Various communications links can be used, such as the Internet, a local area network, a wide area network, or a point-to-point dial-up connection. Thus, computer-readable media can comprise computer-readable storage media (e.g., “non-transitory” media) and computer-readable transmission media.

As used herein, being above a threshold means that a value for an item under comparison is above a specified other value, that an item under comparison is among a certain specified number of items with the largest value, or that an item under comparison has a value within a specified top percentage value. As used herein, being below a threshold means that a value for an item under comparison is below a specified other value, that an item under comparison is among a certain specified number of items with the smallest value, or that an item under comparison has a value within a specified bottom percentage value. As used herein, being within a threshold means that a value for an item under comparison is between two specified other values, that an item under comparison is among a middle specified number of items, or that an item under comparison has a value within a middle specified percentage range.

As used herein, the word “or” refers to any possible permutation of a set of items. For example, the phrase “A, B, or C” refers to at least one of A, B, C, or any combination thereof, such as any of: A; B; C; A and B; A and C; B and C; A, B, and C; or multiple of any item, such as A and A; B, B, and C; A, A, B, C, and C; etc.

The above Detailed Description of examples of the technology is not intended to be exhaustive or to limit the technology to the precise form disclosed above. While specific examples for the technology are described above for illustrative purposes, various equivalent modifications are possible within the scope of the technology. For example, while processes or blocks are presented in a given order, alternative implementations may perform routines having steps, or employ systems having blocks, in a different order, and some processes or blocks may be deleted, moved, added, subdivided, combined, and/or modified to provide alternative or sub-combinations. Each of these processes or blocks may be implemented in a variety of different ways. Also, while processes or blocks are at times shown as being performed in series, these processes or blocks may instead be performed or implemented in parallel, or may be performed at different times. Further, any specific numbers noted herein are only examples: alternative implementations may employ differing values or ranges.

The teachings of the technology provided herein can be applied to other systems, not necessarily the system described above. The elements and acts of the various examples described above can be combined to provide further implementations of the technology. Some alternative implementations of the technology may include not only additional elements to those implementations noted above, but also may include fewer elements.

The description and illustration of one or more aspects provided in this application are not intended to limit or restrict the scope of the disclosure as claimed in any way. The aspects, examples, and details provided in this application are considered sufficient to convey possession and enable others to make and use the best mode of claimed disclosure. The claimed disclosure should not be construed as being limited to any aspect, example, or detail provided in this application. Regardless of whether shown and described in combination or separately, the various features (both structural and methodological) are intended to be selectively rearranged, included or omitted to produce an embodiment with a particular set of features. Having been provided with the description and illustration of the present application, one skilled in the art may envision variations, modifications, and alternate aspects falling within the spirit of the broader aspects of the general inventive concept embodied in this application that do not depart from the broader scope of the claimed disclosure.

What is claimed is:

1. A method comprising:
determining when a reservation period for a light electric vehicle has ended;
initiating, after the reservation period has ended, a sterilization process on a touchpoint of the light electric vehicle, via an internal component; and
providing a notification that the sterilization process is complete.

2. The method of claim 1, wherein the notification that the sterilization process is complete is visual.

3. The method of claim 1, wherein the internal component is a light pipe comprising an ultra-violet light source.

4. The method of claim 3, wherein the light pipe further comprises a light source to provide the notification that the sterilization process is complete.

5. The method of claim 3, wherein the light pipe is at least partially contained within a handlebar tube of the light electric vehicle.

6. The method of claim 5, wherein the handlebar tube of the light electric vehicle includes a slot that permits light from the light pipe to pass through the handlebar tube.

7. The method of claim 1, wherein the touchpoint is a grip portion of a handlebar of the light electric vehicle.

8. The method of 1, further comprising:
providing a notification that the sterilization process is ongoing.

9. The method of claim 8, wherein the notification that the sterilization process is ongoing is visual.

10. A light electric vehicle comprising:
a handlebar including a slot on an exterior surface thereof;
an ultra-violet light source provided within an inner cavity of the handlebar and at least partially aligned with the slot; and
a handlebar grip provided on the handlebar,
wherein the ultra-violet light source initiates a sterilization process on the handlebar grip when illuminated.

11. The light electric vehicle of claim 10, wherein the ultra-violet light source is powered by a rechargeable battery of the light electric vehicle.

12. The light electric vehicle of claim 10, further comprising:
a second light source provided within the inner cavity of the handlebar, the second light source providing a visual indicator that the sterilization process is ongoing.

13. The light electric vehicle of claim 10, further comprising:
a second light source provided within the inner cavity of the handlebar, the second light source providing a visual indicator that the sterilization process is complete.

14. The light electric vehicle of claim 10, further comprising:
a second light source provided within the inner cavity of the handlebar, the second light source providing a visual indicator that the light electric vehicle is performing a turn.

15. The light electric vehicle of claim 10, wherein the handlebar grip includes a cavity that enables light from the ultra-violet light source to be transmitted through the handlebar grip.
